(19) United States
(12) Patent Application Publication
(10) Pub. No.: US 2024/0398859 A1
(43) Pub. Date: Dec. 5, 2024

(54) Stabilized Hydrogen Peroxide

(71) Applicant: CINDRO HOLDING B.V., Andijk (NL)

(72) Inventor: Thomas Hendrik Johannes VERBURG, Twisk (NL)

(21) Appl. No.: 18/694,864

(22) PCT Filed: Sep. 22, 2022
(86) PCT No.: PCT/NL2022/050535

§ 371 Date: Mar. 22, 2024

(30) Foreign Application Priority Data
Sep. 24, 2021 (NL) 2029251

(51) Int. Cl. A61K 33/40; A01N 59/00; A01P 1/00; A61K 47/10; A61K 47/46; C01B 15/037

(52) U.S. Cl. CPC A61K 33/40; A01N 59/00; A01P 1/00; A61K 47/10; A61K 47/46; C01B 15/037

(57) ABSTRACT

The invention relates to hydrogen peroxide stabilized with a coated particle with a diameter of 10⁻⁴-10⁻³ nanometres of one or more antimicrobial plant extracts. These plant extracts are particularly stabilized with a coating of one or more non-toxic alcohols, such as polyols with hydrogen-absorbing properties. The stable hydrogen peroxide according to the invention can be widely applied as disinfectant by removal of bacteria, fungi, viruses, algae, protozoa and/or nematodes. It is for instance possible here to envisage disinfection of surfaces, equipment, air, water and application in human hygiene, in short, any application for which hydrogen peroxide is suitable.

The invention relates to stabilized hydrogen peroxide. The invention further relates to a method for preparation thereof and to an application as disinfectant, biocide, cleaning agent, bleaching agent, crop protection agent, signalling agent and for breaking down crop protection agents and medicines to a maximum residue limit.

In agriculture and horticulture water systems the water contains a great deal of contamination, such as fertilizers, crop protection agents and micro-organisms. These substances form a source of nutrients for a biofilm in which microorganisms can easily settle and develop. A strong disinfectant or cleaning agent is needed to remove this biofilm in long water systems in for instance glass horticulture. In (glass) horticulture, irrigation systems are generally disinfected with biocides with chlorine or hydrogen peroxide as active substances. Chlorine is usually applied in the form of sodium hypochlorite or chlorine dioxide. This water will however come into contact with crops again, and the agent must therefore be present in such low concentration that the biofilm is removed but the crop does not suffer damage.

In order to make this provision hydrogen peroxide is usually applied stabilized with various additives, such as alcohols, sugars and organic acids and silver. The application of alcohols, sugars and organic acids is a good solution for surfaces, since these highly concentrated residues are removed after cleaning. These products are however not applicable in irrigation systems for the following reasons:
excessively aggressive effect on roots,
accumulation of organic acids such as acetic acid, compound with more than 1% carbon, in the system enhances the growth of biofilm,
biofilm creates slime formation, which causes blockages.

In water transport systems these substances are remaining residues which can accumulate in a conduit and then in turn serve as a source of nutrients for microorganisms and biofilm.

Silver-stabilized hydrogen peroxide is an option for solving this problem. Stabilization is required since hydrogen peroxide is not inherently stable, it reacts with organic material, among other things, which causes the hydrogen peroxide particle to break down into water and reactive oxygen compounds (free radicals). The free radical comes into contact with a microorganism, which is oxidized and dies.

The most commonly used silver compounds are water-soluble silver salts. Silver provides for a prolonged action of hydrogen peroxide in low concentration and is therefore highly suitable in irrigation systems for horticulture. The problem with silver (salts) is however that when they are dissolved, toxic nanoparticles are created. These nanoparticles are absorbed by living organisms. Nanoparticles are not filtered in water systems, resulting in exposure to water organisms via discharge of wastewater. Crops can absorb silver nanoparticles via the roots, resulting in exposure to the environment via crop residues, for instance via a recycling process. Finally, silver can be transported in the sap flows of crops to the flowers, fruits and vegetables. This can result in exposure to humans and animals.

Sustainable disinfection of water systems must take place with stabilized hydrogen peroxide, preferably just as stable as silver-stabilized hydrogen peroxide. The additive, or combination of additives, must not be harmful to soil life, crops, humans, animals and the environment. The residue must not enhance the formation of biofilm and the hydrogen peroxide must have long-term effectiveness. Finally, the additive must not be reactive with concentrated hydrogen peroxide. The concentrated final product must have long-term stability, preferably more than 2 years.

The international patent application WO 2010/101844 describes a process wherein plant material reacts with hydrogen peroxide, wherein this compound decomposes into water and oxygen. An antimicrobial oxidizing molecular complex with reactive oxygen is thus formed. There is however a need for hydrogen peroxide which, in concentrate, is stable with a long shelf life and also reacts with biofilm and microorganisms as soon as it is dosed in water systems in low concentrations, without silver (nano) particle residue.

The present invention has for its object to provide stabilized hydrogen peroxide as biocide and disinfectant for preventing and removing microorganisms, especially suitable for disinfecting water transport systems such as pipes (silos, conduits, atomizers, drippers, ebb and flow systems and the like), which is not harmful to the environment.

In order to achieve the stated object the invention provides hydrogen peroxide of the type stated in the preamble, characterized in that it is stabilized with a coated particle of one or more antimicrobial plant extracts. One or more of the plant extracts is preferably selected from the Allium, the Solanaceae, the Magnoliaceae, the Salicaceae, the Cupressaceae and the Chamaecyparis families.

With particular preference, one or more of the plant extracts is selected from Laennecia confusa, Abrus schimperi, Achillea millefolium, Acorus calamus, Aegle marmelos, Agrostemma githago, Allium, for instance Allium cepa and Allium sativum, Aloe barbadensis, Aloe vera, Aloysia triphylla, Anacardium pulsatilla, Anemone pulsatilla, Anethum graveolens, Apocynaceae, Arctium lappa, Armoracia rusticana, Arnica montana, Artemisia dracunculus, Asclepidaceae, Asteraceae, B. citriodora, Barosma setulina, Berberis vulgaris, Bersama engleriana Gurke, Bignoncaceae, Blechnum orientale Linn, Bolusanthus speciosus, Bryophyllum, C. australasica, C. circinalis, C. revoluta, Calendula officinalis, Calpurnia aurea, Camellia sinensis, Cannabis sativa, Capsicum annuum, Carica papaya, Carum carvi, Caryophyllus aromaticus, Cassia angustifolia, Ceasalpinaceae, Centella asiatica, Chamaecyparis formosensis, Chelidonium majus Linn, Cinchona sp, Cinnamomum verum, Citrus paradisa, Citrus sinensis, Clausena heptaphylla, Cocos nucifera, Combretaceae, Convolvulaceae, Coriandrum sativum, Coumarins, Cremaspora triflora, Curcuma longa, Cymbopogon citratus, Echinaceae angustifolia, Elacodendron croceum, Ephedra procera, Erythroxylum coca, Eucalyptus globulus, Euphorbia pulcherrima, Euphorbia tirucalli, Apocynaceae family, Asteraceae family, Combretaceae family, Fabaceae family, Fagaceae family, Labiatae (Lamiaceae) family, Lauraceae family, Loranthaceae family, Myrtaceae family, Piperaceae family, Poaceae family, Polygonaceae family, Ranunculaceae family, Rosaceae family, Rutaceae family, Solanaceae family, Theaceae family, Urticaceae family, Flavones, Flavonoids, Flavonols, Galium odoratum, Garcinia hanburyi, Gaultheria procumbens, Gentianaceae, Gloriosa superba, Glycyrrhiza glabra, Goldenseal (Hydrastis canadensis), Helicanthus elastica, Heteromorpha arborescens, Hibiscus sabdariffa, Holoptelea integrifolia, Humulus lupulus, Hydrangea arborescens, Hydrastis canadensis, Hypericum perforatum, Hypericum roeperianum, Hyssopus officinalis, Jatropha gossyphiifolia, L. ponticum, Lantana camara, Larrea tridentata, Laurus nobilis, Lawsonia, Lawsonia inermis, Lectins, Lemongrass, Liliaceae, Lophophora williamsii, Macsa lanceolata, Magnolia officinalis, Mahonia aquifolia, Malus sylvestris, Matricaria chamomilla, Medicago sativa, Melissa officinalis, Mentha piperita, Mikania glomerata, Millettia thonningii, Momordica charantia, Moraceae, Morus mesozygia, Myristica fragransneem, Ocimum basilicum, Olea europaea, Onobrychis viciifolia, oregano, Panax notoginseng, Papaver somniferum, Peganum harmala, Petalostemum, Pimenta dioica, Piper betel, Piper nigrum, Piper umbellatum, Pittosporum viridiflorum, Platycladus orientalis, Podocarpus nagi, Polygonum aviculare, Premna resinosa, Prosopis juliflora, Psidium guajava, Psidium guineense Swartz, Punica granatum, Pupalia lappacea Juss, Quercus rubra, Quinones, Rabdosia trichocarpa, Ranunculus bulbosus, Rauvolfia serpentina, Rhamnus purshiana, Ricinus communis, Rivea corymbosa, Roeperianum, rosemary, Rosmarinus officinalis, Rumex crispus, Salicaceae, Salix alba, Salvia officinalis, chamaecyparissus, such as for instance Santolina chamaecyparissus, Sassafras albidum, Satureja montana, Schinus terebinthifolius, Scrophulariaceae, Solanum tuberosum, Syzygium aromaticum, Syzygyum joabolanum, T. ferdinandiana, Tabebuia, Tabernaemontana alternifolia, Tanacetum vulgare, Tannins, Taraxacum officinale, Terminalia fagifolia, Terpenoids, Thevetia peruviana, thyme, Thymus vulgaris, tulsi, Tussilago farfara, Vaccinium spp, Valeriana officinalis, Vicia faba, Vinca minor, Withania somniferum, Xanthium strumarium, Zingiber officinale.

Preferably applied are one or more plant extracts with a coating of one or more non-toxic alcohols, preferably polyols with hydrogen-absorbing properties, more preferably selected from polyols with three to ten, preferably three to eight, more preferably three to six hydroxyl groups, particularly glycerol and sorbitol.

A further aspect of the present invention provides a method for preparing stabilized hydrogen peroxide according to the invention, wherein:
one or more antimicrobial plant extracts are mixed with water, preferably in a concentration of 0.0001-1%,
one or more non-toxic alcohols, preferably polyols with hydrogen-absorbing properties, are added thereto under continuous stirring and react at a temperature of 1-70° C., preferably of 50-70° C., more preferably around 60° C., and preferably in a concentration of 0.001-10%,
after mixing 1-70%, preferably 30-50% hydrogen peroxide is added to this mixture and the whole is mixed for 2-4 hours, for instance 3 hours, at room temperature while stirring to form hydrogen peroxide stabilized with a coated particle of one or more of the antimicrobial plant extracts.

If necessary for the solubility of the plant extracts, they are mixed with water in acidic medium at a pH of 0-6, preferably 1-5, particularly 1-3, in the first step. It is then of course possible after this step to dilute the concentrate further until the desired concentrate for filling a flask, bottle, jerrycan, drum, intermediate bulk container or storage tank has been reached.

Another aspect of the invention provides the application of hydrogen peroxide stabilized with a coated particle of one or more plant extracts according to the invention as application as disinfectant, biocide, cleaning agent, bleaching agent, crop protection agent, for counteracting or preventing one or more of the group consisting of bacteria, fungi, viruses, algae, protozoa and nematodes, signalling agent and for breaking down crop protection agents and medicines to a maximum residue limit.

It is particularly favourable to apply stabilized hydrogen peroxide according to the invention
as disinfectant and cleaning agent for removing biofilms in growth media for crops of indoor and outdoor cultivations such as open ground, potting soil and substrates, for removing sources of nutrients on which biofilm, bacteria, fungi and viruses can develop, for cleaning of deposits and organic material on external and internal surfaces,
for treating air, surfaces and water which may or may not be used for human or animal consumption and industrial applications,
for treating waters and surfaces, such as glass, walls, floors, tables and climate screens,
for treating surfaces, materials, equipment and furniture which may or may not be used for direct contact with foodstuffs or animal feed,
for treating equipment, containers, utility articles, conduits which are related to the production, transport, storage or the consumption of food or animal feed and which may or may not be used for direct contact with foodstuffs or animal feeds, including drinking water for humans or animals,
for human hygiene, applied to or in contact with the human skin or scalp with the main objective of sanitizing the skin or scalp, for veterinary hygiene, products for oral or body hygiene or with an antimicrobial action, for both internal and external use, such as on wounds, in the mouth, stomach and intestines; all this particularly by means of periodic and continuous dosing, immersion, spraying, atomizing or fogging.

The fields of application of the stable hydrogen peroxide according to the invention particularly comprise inter alia:
source of oxygen addition for enriching soil and growth media for cultivation of agricultural and horticultural crops,
source of oxygen addition to drinking water for humans and animals,
source of oxygen addition to pools, spas, sauna, cosmetic treatments, showers,
enriching water and water systems-such as in agriculture and horticulture, hydroculture and vertical agriculture-with additional dissolved oxygen,
treating (drinking) water and (drinking) water systems for animals,
treating conduits and components of milking equipment and milk tanks,
treating veterinary applications, such as spaces where animals are treated, kept, processed or transported, such as animal transport means,
treating surfaces, spaces, materials, water systems and (medical) aids and other objects or living organisms which may be a vector/carrier of microorganisms and viruses such as COVID,
treating organic material, such as manure, as part of a production process of an energy source or to treat less harmful nitrogen compounds or separate them from compounds,
treating water in storage tanks and the associated water systems for the purpose of reducing potentially toxic substances such as nitrite;
in short, any field of application for which hydrogen peroxide is suitable.

Hydrogen peroxide stabilized with a coated particle of one or more plant extracts according to the invention for use as medicine or medical aid in internal or external medical treatments also forms part of the invention.

Stable hydrogen peroxide according to the invention can also be incorporated in textiles, tissues, masks, paints and other articles or materials with the object of producing treated articles with disinfectant properties.

Hydrogen peroxide reacts with most organic substances. Surprisingly, we have found that antimicrobial plant extracts are able to form a bond with hydrogen peroxide under determined conditions, whereby the hydrogen peroxide particle ‘decomposes’ less readily. It was furthermore highly surprising that more dissolved oxygen is measured, while less hydrogen peroxide has decomposed. This means that, once dissolved in the irrigation water, the stabilizers also enhance a stabilization of oxygen particles.

Long-term stabilization takes place and a mild reaction results with harmful organisms in complex water systems, such as microorganisms (bacteria, fungi, viruses) and biofilms. This has the result that sustainable irrigation water with healthy dissolved oxygen contents is created, that development of the soil life (such as enzymes) is enhanced and healthy crop development is supported by formation of vital roots, root hairs and root tips. No exposure to harmful residues takes place here.

Stabilized hydrogen peroxide according to the invention is a solution of hydrogen peroxide with a coated particle of one or more antimicrobial plant extracts, preferably in a concentration of 0.1-70%. Stabilized hydrogen peroxide has the property that it is more stable in contaminated water in low concentrations in the range of 1-100 ppm—such as irrigation water—and is more effective against microorganisms, and keeps oxygen in solution in water systems for longer.

Hydrogen peroxide stabilized with a coated particle of one or more plant extracts according to the invention provides a sufficient solution to a long-standing problem: providing a disinfectant which is not harmful to the environment and does not leave behind a residue, with a long-lasting stability of at least 2 years with a peroxide reduction of a maximum of 2% per year of a hydrogen peroxide 50% concentrate.

In commercial packaging the hydrogen peroxide must have a high concentration, preferably between 0.1-70%. The most common concentrate for major consumers in the agricultural sector will be 30-50%. Ready-made concentrates which are directly applicable for treatment of surfaces will mainly contain hydrogen peroxide in concentration of 1-8%. The product can be packaged in spray bottles (+/−100-1000 cc), bottles (+/−11), jerrycans (+/−5, 10, 20 1), drums (up to +/−200 1) and intermediate bulk containers (+/−1000 1). The additive-or combination of additives-will preferably be present in a concentration of 0.01-10%, most preferably between 0.01 and 1%, this being the case for highly-concentrated stabilized hydrogen peroxide 50%; proportionally, these concentrations will be higher or lower in other peroxide concentrations. Application by means of continuous dosing in water systems will take place in a concentration of mainly 1-100 ppm, with exceptions of up to as much as 1000 ppm. Shock dosing in water systems takes place mainly in concentrations of 0.5%-6%, with a contact time of multiple hours for 100% disinfection. In atomizing applications a concentration of hydrogen peroxide in the air of 10-1000 ppm is usually achieved, in large greenhouses this range often lies around 10-100 ppm and in closed spaces such as laboratories or medical institutions the range often lies between 50-600 ppm. In application as spray on crops the concentration range is about 0.1-3%, usually 0.5-1%. In surface applications the range is often between 1-15%, mainly 1-6%. Said concentrations are the hydrogen peroxide content. Most applications will take place in agriculture and horticulture (indoor cultivations, outdoor cultivations, hydroculture, vertical agriculture, breeding, seed treatment, application on crops, in water systems, surface treatment of cultivation systems, greenhouse disinfection, maintenance/cleaning/disinfection of climate screens, root treatment, substrate treatment, breaking down of crop protection agents, cleaning of filters, and so on), food processing industry, veterinary field, medical field, shipping, aviation, hospitality industry, logistics industry, public spaces, amusement parks and rides, (play) fountains, shops, supermarkets, petrol stations, sanitary spaces, government agencies, permanent or temporary dwellings for animals and humans, sports facilities. Applications take place mostly by means of spraying onto surfaces, atomizing in air/spaces and dosing in water systems to, among other things, counteract/prevent (cross-)contamination by bacteria, fungi and viruses, remove biofilm and organic material and other contamination/deposits and add oxygen to water or soil and improve microbiological and enzymatic life in or on bodies of humans and animals or soil/growth media for the cultivation of crops.

In the present application coated particle is understood to mean a coated particle with a diameter of 10⁻⁴-10⁻³ nanometres. In the present application antimicrobial is understood to mean active against microorganisms such as bacteria (including vegetative cells, spores and mycobacteria), fungi (including yeasts), and viruses (including bacteriophages). Biofilm is understood to mean a layer of microorganisms surrounded by self-produced slime or slime created by organic contamination, adhered to a surface. Hydrogen peroxide stabilized with a coated particle of one or more antimicrobial plant extracts according to the invention is also referred to as bio-stabilized hydrogen peroxide. The maximum residue limit (MRL) is the maximum amount of a crop protection agent or medicine which is permitted to be present in food.

The invention is elucidated on the basis of the following examples.

### EXAMPLE 1. PREPARATION OF HYDROGEN PEROXIDE WITH GARLIC AND PEPPER EXTRACT

100 g of garlic extract and 500 g of pepper extract were mixed in 1000 ml of demineralized water; the pH was brought to 2 by adding nitric acid. 2000 ml of glycerol was added under continuous stirring and this mixture was heated to 60° C. The whole was mixed at 1100 rpm for 6 hours with an overhead stirrer.

This whole mixture was then added to 1000 1 of 35% hydrogen peroxide. The whole was then mixed at 1500 rpm at room temperature for 3 hours. This results in 1000 litres of 35% hydrogen peroxide stabilized with coated particles of garlic and pepper extract. This mixture is stable for at least 2 years. This product can be dosed in water systems for removal of biofilm and as disinfectant by removal of bacteria, fungi, viruses, algae, protozoa and/or nematodes.

### EXAMPLE 2. PREPARATION OF HYDROGEN PEROXIDE WITH MAGNOLIA OFFICINALIS EXTRACT AND PLATYCLADUS ORIENTALIS EXTRACT

200 g of Magnolia officinalis extract and 300 g of Platycladus orientalis extract were mixed in 2000 ml of demineralized water; the pH was brought to 2 by adding phosphoric acid. 1000 ml of sorbitol and 1000 ml of glycerol were added under continuous stirring and this mixture was heated to 60° C. The whole was mixed at 1100 rpm for 6 hours with an overhead stirrer.

This whole mixture was then added to 1000 1 of 50% hydrogen peroxide. The whole was then mixed at 1500 rpm at room temperature for 3 hours. This results in 1000 litres of 50% hydrogen peroxide stabilized with coated particles of Magnolia officinalis extract and Platycladus orientalis extract. This mixture is stable for at least 2 years. This product can be dosed in water systems for removal of biofilm and as disinfectant by removal of bacteria, fungi, viruses, algae, protozoa and/or nematodes.

### EXAMPLE 3. PREPARATION OF HYDROGEN PEROXIDE WITH WHITE WILLOW BARK EXTRACT

100 g of white willow bark extract was mixed in 2000 ml of demineralized water. 1000 ml of polyethylene glycol was added under continuous stirring. The whole was mixed at 1100 rpm for 6 hours with an overhead stirrer.

This whole mixture was then added to 1000 1 of 50% hydrogen peroxide. The whole was then mixed at 1500 rpm at room temperature for 3 hours. This results in 1000 litres of 50% hydrogen peroxide stabilized with coated particles of white willow bark extract. This mixture is stable for at least 2 years, with a peroxide reduction of a maximum of 2% per year. This product can be dosed in water systems as oxidizing cleaning agent for removal of minerals, deposits, biofilm and as disinfectant by removal of bacteria, fungi, viruses, algae, protozoa and/or nematodes, as crop protection agent by means of spraying onto plants, for breaking down crop protection agent residues and as a source of oxygen for water.

### EXAMPLE 4. COMPARISON OF EFFECT OF SILVER STABILIZED WITH BIO-STABILIZED HYDROGEN PEROXIDE (AS PREPARED IN EXAMPLE 1, 2 or 3)

In a six-month period three identical water systems with bacteria and fungi are followed in parallel: untreated water system, water system with silver-stabilized hydrogen peroxide and with bio-stabilized hydrogen peroxide.

In week 18 the test site was closed for Christmas and New Year's Eve, allowing a pump defect to impact the dosing. The dosage was 40 ml/m³ via a pulse pump and coupled to a water meter. Measurements every 7 days: concentration of hydrogen peroxide shown in mg/l, bacteria in colony-forming units (CFU) and dissolved oxygen (%); the start of the irrigation system lies in the ‘fertilization unit’, the end of the irrigation system lies at the ‘crops’.

### Results

### Untreated Control

A low dissolved oxygen content (64-91%) inhibits root development. A high germ count of bacteria, i.e. higher than 100,000 CFU, enhances biofilm growth: an elevated formation of biofilm stimulates deposition of nutrients. This water is not suitable as irrigation water for healthy crops.

### Silver-Stabilized Hydrogen Peroxide

This water system was cleaner at the start compared to the water system treated with bio-stabilized hydrogen peroxide. The biofilm was removed quickly (week 3). After the fertilizer unit the concentration of hydrogen peroxide decreased relatively rapidly. The level of dissolved oxygen is higher compared to untreated hydrogen peroxide because the formation of biofilm is zero. The concentration of bacteria is low and oxygen is released by reaction of the hydrogen peroxide.

The agent is effective in preventing biofilm.

### Bio-Stabilized Hydrogen Peroxide

The system was very dirty at the start, but by week 6 the system was comparable to silver-stabilized hydrogen peroxide.

The ratio of the concentration of hydrogen peroxide, bacteria in CFU and dissolved oxygen is notable. The stability of hydrogen peroxide is higher at the fertilization unit and crops, while the same amount of CFU are measured compared to silver-stabilized hydrogen peroxide. This means that less product is needed to control the CFU, this being a sign of increased stability since the product reacts only when necessary, and does not decompose on its own. In addition, the level of dissolved oxygen is higher on average when decomposition of hydrogen peroxide is lower; this also being a sign of stability of oxygen particles in water. The hydrogen peroxide and the dissolved oxygen remain in solution in the irrigation water better in the presence of bio-stabilizer as compared to stabilization by means of silver particles.

The agent is highly effective in preventing biofilm.

### IN CONCLUSION

Bio-stabilized hydrogen peroxide is effective in preventing and removing biofilm. This controls the development of bacteria and creates a high dissolved oxygen content in water. Surprisingly, no increase of biofilm took place due to possible accumulation of biological substances of the stabilizers. It is suspected that it is converted into carbon, which is available to the plant as nutrient. Bio-stabilized hydrogen peroxide is more stable than silver-stabilized hydrogen peroxide, without the risk of exposure to heavy metals.

Although the invention has been elucidated above with reference to a number of preparations and application of bio-stabilized hydrogen peroxide, it will be apparent that the invention is by no means limited thereto. On the contrary, many variations are possible within the scope of the invention for a person with ordinary skill in the art.

1. Hydrogen peroxide stabilized with a coated particle of one or more antimicrobial plant extracts.

2. Hydrogen peroxide according to claim 1, stabilized with one or more plant extracts selected from the Allium, the Solanaceae, the Magnoliaceae, the Salicaceae, the Cupressaceae and the Chamaecyparis families.

3. Hydrogen peroxide according to claim 1 or 2, stabilized with plant extracts with a coating of one or more non-toxic alcohols, preferably polyols with hydrogen-absorbing properties.

4. Hydrogen peroxide according to claim 3, stabilized with plant extracts with a coating of one or more polyols selected from polyols with three to ten, preferably three to eight hydroxyl groups.

5. Method for preparing stabilized hydrogen peroxide according to one or more of the foregoing claims, wherein:
one or more antimicrobial plant extracts are mixed with water, preferably in a concentration of 0.0001-1%,
one or more non-toxic alcohols, preferably polyols with hydrogen-absorbing properties, are added thereto under continuous stirring and react at a temperature of 1-70° C., preferably in a concentration of 0.001-10%,
after mixing 1-70%, preferably 30-50% hydrogen peroxide is added to this mixture and the whole is mixed for 2-4 hours at room temperature while stirring to form hydrogen peroxide stabilized with a coated particle of one or more of the antimicrobial plant extracts.

6. Application of hydrogen peroxide stabilized with a coated particle of one or more plant extracts according to one or more of the claims 1-4 as application as disinfectant, biocide, cleaning agent, bleaching agent, crop protection agent, for counteracting or preventing one or more of the group consisting of bacteria, fungi, viruses, algae, protozoa and nematodes, signalling agent and for breaking down crop protection agents and medicines to a maximum residue limit.

7. Application of hydrogen peroxide according to claim 6,
as disinfectant and cleaning agent for removing biofilms in growth media for crops of indoor and outdoor cultivations such as open ground, potting soil and substrates, for removing sources of nutrients on which biofilm, bacteria, fungi and viruses can develop, for cleaning of deposits and organic material on external and internal surfaces,
for treating air, surfaces and water which may or may not be used for human or animal consumption and industrial applications,
for treating waters and surfaces, such as glass, walls, floors, tables and climate screens,
for treating surfaces, materials, equipment and furniture which may or may not be used for direct contact with foodstuffs or animal feed,
for treating equipment, containers, utility articles, conduits which are related to the production, transport, storage or the consumption of food or animal feed and which may or may not be used for direct contact with foodstuffs or animal feeds, including drinking water for humans or animals,
for human hygiene, applied to or in contact with the human skin or scalp with the main objective of sanitizing the skin or scalp, for veterinary hygiene, products for oral or body hygiene or with an antimicrobial action, for both internal and external use, such as on wounds, in the mouth, stomach and intestines;
all this particularly by means of periodic and continuous dosing, immersion, spraying, atomizing or fogging.

8. Application of hydrogen peroxide stabilized with a coated particle of one or more plant extracts according to one or more of the claims 1-4,
as source of oxygen addition for enriching soil and growth media for cultivation of agricultural and horticultural crops,
as source of oxygen addition to drinking water for humans and animals,
as source of oxygen addition to pools, spas, sauna, cosmetic treatments, showers,
for enriching water and water systems—such as in agriculture and horticulture, hydroculture and vertical agriculture—with additional dissolved oxygen,
for treating (drinking) water and (drinking) water systems for animals,
for treating conduits and components of milking equipment and milk tanks,
for treating veterinary applications, such as spaces where animals are treated, kept, processed or transported, such as animal transport means,
for treating surfaces, spaces, materials, water systems and (medical) aids and other objects or living organisms which may be a vector/carrier of microorganisms and viruses such as COVID,
for treating organic material, such as manure, as part of a production process of an energy source or to treat less harmful nitrogen compounds or separate them from compounds,
for treating water in storage tanks and the associated water systems for the purpose of reducing potentially toxic substances such as nitrite.

9. Hydrogen peroxide stabilized with a coated particle of one or more plant extracts according to one or more of the claims 1-4, for use as medicine or medical aid in internal or external medical treatments.
